(19) United States
(12) Patent Application Publication
(10) Pub. No.: US 2022/0379104 A1
(43) Pub. Date: Dec. 1, 2022

(54) Vascular Access Device Having an Instrument Configured to Insert Into a Catheter

(71) Applicant: Becton, Dickinson and Company, Franklin Lakes, NJ (US)

(72) Inventors: Curtis H. Blanchard, Riverton, UT (US); Weston F. Harding, Lehi, UT (US); Yiping Ma, Layton, UT (US)

(21) Appl. No.: 17/752,375

(22) Filed: May 24, 2022

Related U.S. Application Data
(60) Provisional application No. 63/193,478, filed on May 26, 2021.

(51) Int. Cl. A61M 39/02; A61M 25/09

(52) U.S. Cl. CPC A61M 39/0247; A61M 25/09041; A61M 2039/0258; A61M 2039/027; A61M 2039/0282; A61M 2039/0288

(57) ABSTRACT

A vascular access device may include a housing, which may include a distal end, a proximal end, and a lumen extending through the distal end of the housing and the proximal end of the housing. The distal end of the housing may include a connector configured to couple to a catheter assembly. The vascular access device may include a bag, which may be coupled to the proximal end of the housing. The vascular access device may include an instrument and a pincher disposed within the bag. The pincher may be configured to pinch the instrument to move the instrument distally through the lumen of the housing and into the catheter assembly.

### CROSS-REFERENCE TO RELATED APPLICATION

The present application claims priority to U.S. Provisional Application Ser. No. 63/193,478, entitled “Vascular Access Device Having an Instrument Configured to Insert into a Catheter”, filed May 26, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increase. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related methods. In some embodiments, a vascular access device may include a housing, which may include a distal end, a proximal end, and a lumen extending through the distal end of the housing and the proximal end of the housing. In some embodiments, the distal end of the housing may include a connector configured to couple to a catheter assembly.

In some embodiments, the vascular access device may include a bag coupled to the proximal end of the housing. In some embodiments, the vascular access device may include an instrument disposed within the bag. In some embodiments, the instrument may include a distal end and a proximal end. In some embodiments, the vascular access device may include a pincher disposed within the bag and configured to pinch the instrument to move the instrument distally through the lumen of the housing.

In some embodiments, the vascular access device may include a septum, which may be disposed within the housing or a distal end of the bag. In some embodiments, the vascular access device may include a pull cord, which may be coupled to the proximal end of the instrument and may extend through an opening in the bag. In some embodiments, a proximal end of the pull cord may be coupled to a grip element.

In some embodiments, the housing may include a port between the distal end of the housing and the proximal end of the housing. In some embodiments, the vascular access device may include an extension tube and a blood collection device. In some embodiments, the extension tube may include a distal end integrated with the port and a proximal end coupled to the blood collection device.

In some embodiments, the blood collection device may include a sharp cannula. In some embodiments, the instrument may include a guidewire. In some embodiments, the pincher may include a U-shaped body having two arms configured to move towards each other to pinch the instrument. In some embodiments, an inner surface of the two arms may include a textured surface, such as a rough surface or an uneven surface, for example.

In some embodiments, the vascular access device may include a position indicator, which may be disposed on the instrument. In some embodiments, the instrument is in a retracted position. In some embodiments, in response to the instrument being in the retracted position, the distal end of the instrument may be disposed within the housing and the position indicator is disposed within the bag. In some embodiments, the pull cord may be compacted in response to the instrument being in the retracted position.

In some embodiments, the vascular access device may include a stop disposed on the instrument with an enlarged outer diameter. In some embodiments, in response to the instrument moving distally from the retracted position to an advanced position distal to the housing, the stop may contact an inner surface of the housing.

In some embodiments, a method may include coupling the vascular access device to the catheter assembly. In some embodiments, the method may include pinching the instrument with the pincher after coupling the vascular access device to the catheter assembly. In some embodiments, after pinching the instrument with the pincher, the method may include moving the instrument from the retracted position to the advanced position. In some embodiments, in response to the instrument being in the retracted position, the distal end of the instrument may be within the housing. In some embodiments, in response to the instrument being in the advanced position, the distal end of the instrument may be disposed within the catheter assembly or distal to the catheter assembly.

In some embodiments, the method may include collecting blood through the catheter assembly and the vascular access device after moving the instrument from the retracted position from the advanced position. In some embodiments, the method may include moving the instrument from the advanced position to the retracted position after collecting the blood through the catheter assembly and the vascular access device. In some embodiments, moving the instrument from the advanced position to the retracted position may include pulling the pull cord proximally.

In some embodiments, moving the instrument from the advanced position to the retracted position may include moving the distal end of the instrument from a position distal to the bag into the bag. In some embodiments, in response to the instrument being in the retracted position, the distal end of the instrument may be disposed within the housing and the position indicator is disposed within the bag.

In some embodiments, the vascular access device and movement of instrument to the advanced position may facilitate patency, fluid infusion, and blood draw by preventing or delaying thrombus development and attachment to a catheter or another portion of the catheter assembly.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example vascular access device, illustrating an example instrument in an example retracted position, according to some embodiments;

FIG. 1B is an enlarged upper perspective view of the vascular access device, illustrating the instrument in the retracted position, according to some embodiments;

FIG. 1C is an upper perspective view of the vascular access device, illustrating the instrument in an example advanced position, according to some embodiments;

FIG. 2 is an upper perspective view of an example catheter system, illustrating the instrument in the retracted position, according to some embodiments;

FIG. 3 is a cross-sectional view of the catheter system, illustrating the instrument in the advanced position, according to some embodiments;

FIG. 4A is an upper perspective view of the catheter system inserted into vasculature of a patient, according to some embodiments;

FIG. 4B is an upper perspective view of the catheter system, illustrating a user pinching an example pincher to move the instrument to the advanced position, according to some embodiments

FIG. 4C is an upper perspective view of the catheter system, illustrating the user collecting blood, according to some embodiments;

FIG. 4D is an upper perspective view of the catheter system, illustrating the user pulling an example pull cord in a proximal direction to move the instrument to the retracted position, according to some embodiments;

FIG. 5A is an upper perspective view of the pincher, according to some embodiments;

FIG. 5B is a lower perspective view of the pincher, according to some embodiments;

FIG. 5C is a lower perspective view of the pincher, illustrating the instrument extending therethrough, according to some embodiments; and

FIG. 5D is a lower perspective view of the pincher, illustrating the pincher in a closed position pinching the instrument, according to some embodiments.

### DETAILED DESCRIPTION OF THE INVENTION

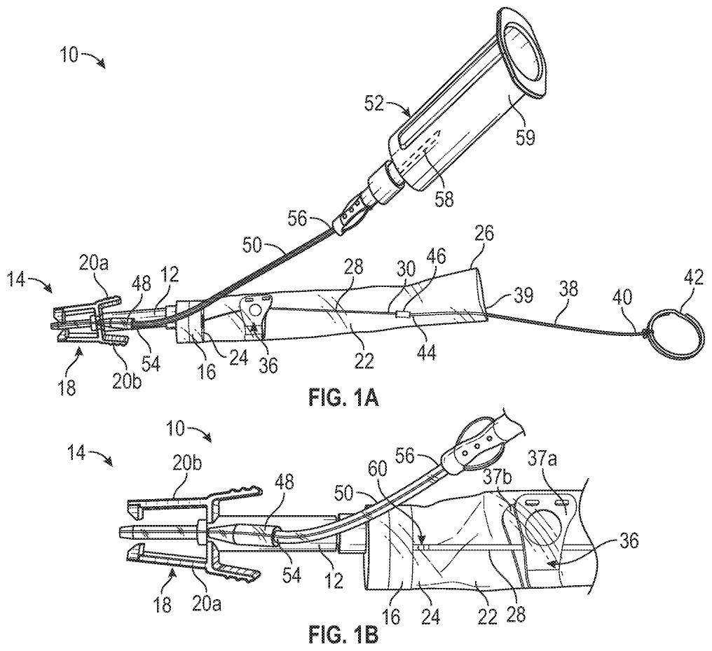

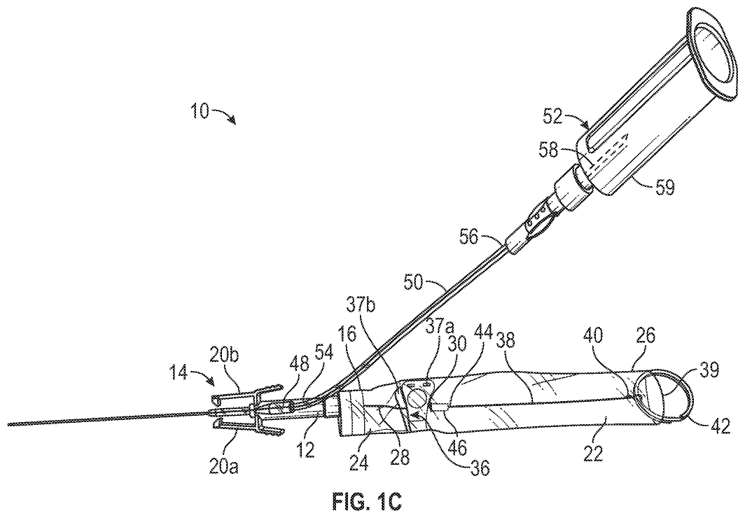

Referring now to FIGS. 1A-1C, in some embodiments, a vascular access device 10 may include a housing 12, which may include a distal end 14, a proximal end 16, and a lumen extending through the distal end 14 of the housing 12 and the proximal end 16 of the housing 12. In some embodiments, the distal end 14 of the housing 12 may include a connector 18 configured to couple to a catheter assembly.

In some embodiments, the connector 18 may include a male luer adapter or another suitable connector. In some embodiments, the connector 18 may include opposing lever arms 20a,20b. In some embodiments, distal ends of the opposing lever arms 20a,20b may be configured to move apart from each other in response to pressure applied to proximal ends of the opposing lever arms 20a,20b. In some embodiments, in response to removal of the pressure applied to the proximal ends of the opposing lever arms 20a,20b, the distal ends may move closer to each other and clasp a portion of the catheter assembly, such as a needleless connector, a proximal end of a catheter adapter, or another portion of the catheter assembly.

In some embodiments, the vascular access device 10 may include a bag 22, which may be coupled to the proximal end 16 of the housing 12. In some embodiments, the bag 22 may include a distal end 24 and a proximal end 26. In some embodiments, the distal end 24 of the bag 22 may be sealed around the proximal end 16 of the housing 12. In some embodiments, the bag 22 may be approximately 6 inches long or another suitable length. In some embodiments, the housing 12 may be monolithically formed as a single unit. In other embodiments, the housing 12 may include multiple pieces coupled together.

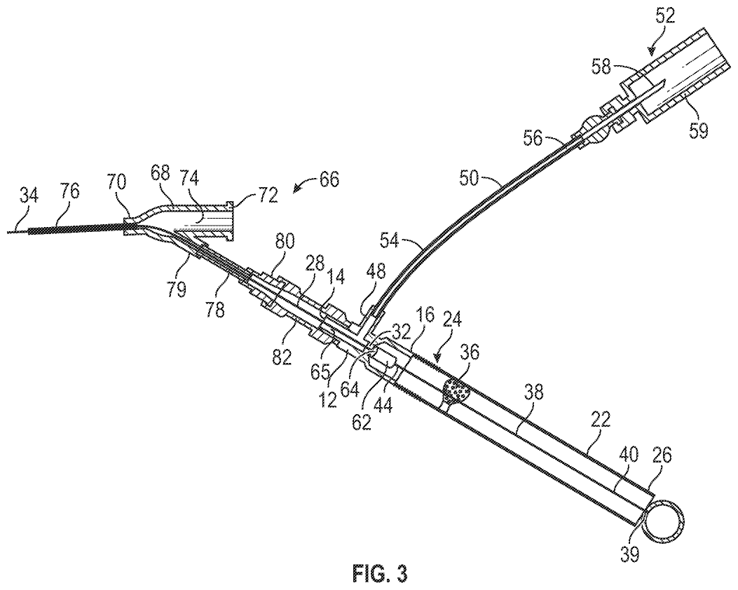

In some embodiments, the vascular access device 10 may include an instrument 28 disposed within the bag 22, which may protect the instrument 28 from bacteria. In some embodiments, the instrument 28 may be fully disposed within the bag 22 or partially disposed within the bag 22. In some embodiments, a proximal end 30 of the instrument 28 may be disposed within the bag 22 when the instrument 28 is in an advanced position and/or a retracted position. In some embodiments, the vascular access device 10 may include a septum 32 (see, for example, FIG. 3), which may be disposed within the housing 12 or the distal end 24 of the bag 22. In some embodiments, a distal end of the instrument 28 may extend through the septum 32 and/or be disposed within the housing 12 when the instrument 28 is in the retracted position. In some embodiments, the septum 32 may provide guidance to the instrument 28 as the instrument 28 moves from the retracted position to the advanced position. In some embodiments, the septum 32 may provide some drag that may facilitate maintaining the instrument 28 in a desired position.

In some embodiments, the instrument 28 may include a guidewire, a probe, a guidewire or a probe with one or more sensors, or another suitable instrument. In some embodiments, the sensors may be used for patient or device monitoring and may include sensors measuring pressure, temperature, pH, blood chemistry, oxygen saturation, flow rate, or another physiological measure. In some embodiments, the instrument 28 may improve patency of the catheter of the catheter assembly for medication and fluid delivery, as well as blood acquisition, through a dwell time of the catheter.

In some embodiments, the instrument 28 may be constructed of metal or another suitable material. In some embodiments, the instrument 28 may be generally linear. In some embodiments, the instrument 28 may extend a life of the catheter for infusion and/or blood collection. For example, the instrument 28 may be configured to reduce or remove a thrombus from within the catheter assembly, within a catheter of the catheter assembly, or at a distal tip of the catheter of the catheter assembly. In some embodiments, the instrument 28 may open a valve within the vasculature and/or move obstructions away from the distal tip of the catheter.

In some embodiments, the vascular access device 10 may include a pincher 36 disposed within the bag 22 and configured to pinch the instrument 28 to move the instrument 28 distally through the lumen of the housing 12. In some embodiments, the pincher 36 may include a U-shaped body having two arms 37a,37b configured to move towards each other to pinch the instrument 28. In some embodiments, an inner surface of the two arms 37a,37b may include a textured surface, which may enhance grip of the instrument 28 and prevent slipping. For example, the inner surface of the two arms 37a,37b may include an uneven surface with multiple bumps or protrusions or a rough surface.

In some embodiments, the vascular access device 10 may include a pull cord 38, which may be coupled to the proximal end 30 of the instrument 28 and may extend through an opening 39 in the bag 22. In some embodiments, the pull cord 38 may include string, rope, an elastomer, or another suitable material.

In some embodiments, a proximal end 40 of the pull cord 38 may be coupled to a grip element 42. In some embodiments, the grip element 42 may include a tab, a ring, or another suitable grip element configured to facilitate gripping by a user. In some embodiments, a distal end 44 of the pull cord 38 may be tied or bonded to the proximal end 30 of the instrument 28 or coupled to the proximal end 30 of the instrument 28 via a connector 46 or another suitable method. In some embodiments, the proximal end 30 of the instrument 28 and/or the distal end 44 of the pull cord 38 may be integrated into the connector 46.

In some embodiments, the housing 12 may include a port 48 between the distal end 14 of the housing 12 and the proximal end 16 of the housing 12. In some embodiments, the vascular access device 10 may include an extension tube 50 and a blood collection device 52. In some embodiments, the extension tube 50 may include a distal end 54 integrated with the port 48 and a proximal end 56 coupled to the blood collection device 52. In some embodiments, the extension tube 50 may be in fluid communication with the lumen of the housing 12.

In some embodiments, the blood collection device 52 may include a sharp cannula 58 and/or a holder 59 configured to receive a container such as a test tube or BD VACUTAINER® Blood Collection Tube, available from Becton Dickinson & Company of Franklin Lakes, N.J. In some embodiments, the blood collection device 52 may include a luer lock access device or another suitable device.

In some embodiments, the instrument 28 may be in a retracted position, as illustrated, for example in FIGS. 1A-1B. In some embodiments, the instrument 28 may initially be placed in the retracted position during manufacture and/or the instrument 28 may be moved to the retracted position after being in the advanced position, illustrated, for example, in FIG. 1C. In some embodiments, the vascular access device 10 may include a position indicator 60, which may be disposed on the instrument 28. In some embodiments, the position indicator 60 may include a marking on the instrument 28, such as, for example, one or more lines or one or more annular lines. In some embodiments, the position indicator 60 may include a different color than portions of the instrument 28 proximate or adjacent the position indicator 60, which may facilitate visualization of the position indicator 60. In some embodiments, the bag 22 may be transparent, which may facilitate visualization of the position indicator 60 by the user.

In some embodiments, in response to the instrument 28 being in the retracted position, the distal end 24 of the instrument 28 may be disposed within the housing 12 and/or the position indicator 60 may be disposed within the bag 22. Thus, in some embodiments, the position indicator 60 may indicate to the user that the instrument 28 is in the retracted position. In some embodiments, in response to the instrument 28 being in the retracted position, the position indicator 60 may be approximately aligned with the proximal end 16 of the housing 12. In some embodiments, the bag 22 and/or the housing 12 may include another position indicator, and in response to the position indicator 60 being aligned with the other position indicator, the instrument 28 may be in the retracted position or another position. In some embodiments, the other position indicator may be similar or identical to the position indicator 60 in terms of one or more features. In some embodiments, the pull cord 38 may be compacted within the bag 22 in response to the instrument 28 being in the retracted position. For example, the pull cord 38 may be wound or folded back on itself within the bag 22.

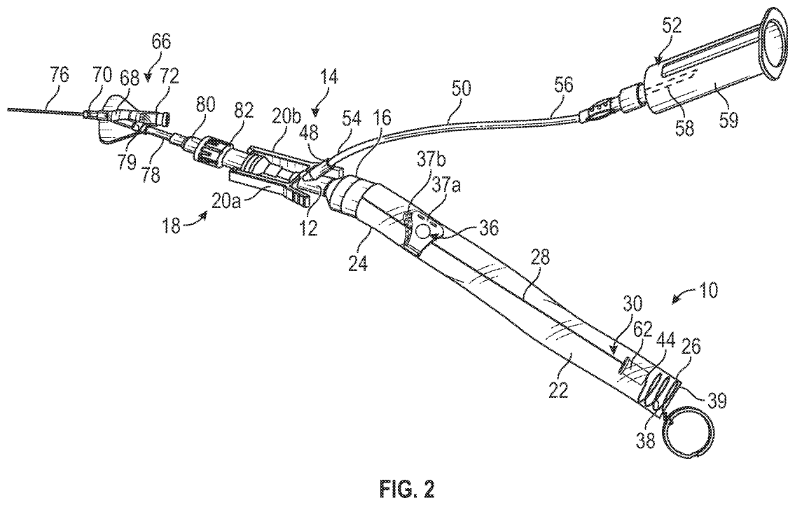

Referring now to FIGS. 2-3, in some embodiments, an outer diameter of the pull cord 38 may be approximately equal to or slightly less than an inner diameter of the opening 39 such that the opening 39 forms a seal around the pull cord 38 or air flowing through the opening 39 is limited, which may decrease a risk of bacterial contamination or the vascular access device 10 and the instrument 28. In some embodiments, the opening 39 may prevent the bag 22 from pinching and forcing air into the patient. In some embodiments, the bag 22 may include the opening 39, another suitable opening, a vent, a membrane, or another suitable feature through which air and/or the pull cord 39 may pass.

In some embodiments, the vascular access device 10 may include a stop 62 disposed on the instrument 28 with an enlarged outer diameter. In some embodiments, in response to the instrument 28 moving distally from the retracted position to the advanced position, the stop 62 may contact an inner surface 64 of the housing 12 and/or the septum 32, and the instrument 28 may be prevented from advancing further in a distal direction. In some embodiments, the inner surface 64 may be disposed within the lumen 65 of the housing 12. In some embodiments, the stop 62 may correspond to the connector 46.

In some embodiments, the vascular access device 10 may be coupled to the catheter assembly 66. In some embodiments, the catheter assembly 66 may include the catheter adapter 68, which may include a distal end 70, a proximal end 72, and a lumen 74 extending through the distal end 70 and the proximal end 72. In some embodiments, the catheter assembly 66 may include the catheter 76, which may extend distally from the catheter adapter 68. In some embodiments, the catheter 76 may include a peripheral intravenous catheter, a midline catheter, or a peripherally inserted central catheter. In some embodiments, a needle assembly (not illustrated) may be coupled to the proximal end 72 of the catheter adapter 68 and may include an introducer needle extending through the catheter 76. In some embodiments, the needle assembly may be removed from the catheter assembly 66 in response to the catheter 76 being positioned within vasculature of a patient and/or observation of flashback of blood.

In some embodiments, the catheter adapter 68 may include a side port 79 disposed between the distal end 70 and the proximal end 72 of the catheter adapter 68. In some embodiments, a distal end of an extension tube 78 may be integrated with the side port 79 and/or a proximal end of the extension tube 78 may be integrated with a connector 80. In some embodiments, the connector 80 may be coupled with a needleless connector 82, which may be coupled to the vascular access device 10. In some embodiments, the catheter assembly 66 and its components may vary. In some embodiments, the vascular access device 10 may be connected to another portion of the catheter assembly 66.

In some embodiments, in response to insertion of the catheter 76 into the vasculature of the patient, blood may flow in a fluid pathway that may extend through one or more of the catheter 76, the catheter adapter 68, the side port 79, the connector 80, and the needleless connector 82. In some embodiments, the fluid pathway may extend into the housing 12, through the port 48, and to the blood collection device 52. In some embodiments, the blood may be prevented from flowing proximally through the housing 12 and into the bag 22 due to presence of the septum 32, which may provide a fluid seal. In some embodiments, the septum 32 may include any suitable septum configured to provide the fluid seal.

In some embodiments, a length L of a portion of the fluid pathway within the vascular access device 10 may be selected based on one or more of the following: a gauge of the catheter 76, a particular configuration of the catheter assembly 66, or a clinical setup. In some embodiments, the portion of the fluid pathway within the vascular access device 10 may extend from the distal end 14 of the housing 12 through the port 48 and through the extension tube 50. In some embodiments, the portion of the fluid pathway may extend through a connector disposed at a proximal end of the extension tube 50 and/or into the blood collection device 52, such as, the sharp cannula 58. In some embodiments, the portion of the fluid pathway may include an inner diameter D.

Fluid flow in the portion of the fluid pathway can be analyzed using Poiseuille's equation, when the portion of the fluid pathway is tubular:

$$Q = \frac{π {D}^{4} Δ P}{128 μ L} = \frac{Δ P}{{R}_{f}}$$

where ΔP is a change in pressure gradient across the length of the portion of the fluid pathway, D and L are the inner diameter and length, respectively, of the portion of the fluid pathway, μ is the viscosity of a fluid, and

$${R}_{f} = \frac{128 μ L}{π {D}^{4}}$$

is the fluid resistance. Since μ is the viscosity of the fluid and not part of the extension tube geometry, a geometric factor G_{f} is defined such that R_{f} (the fluid resistance) is

$${R}_{f} = \frac{128 μ}{π} {G}_{f} , where {G}_{f} = \frac{L}{{D}^{4}} .$$

In some embodiments, the portion of the fluid pathway may have multiple sections with lengths (L1, L2, L3) and inner diameters of (D1, D2, D3), the geometric factor is then:

$${G}_{f} = \frac{L 1}{D {1}^{4}} + \frac{L 2}{D {2}^{4}} + \frac{L 3}{D {3}^{4}}$$

In some embodiments, the portion of the fluid pathway may have an inner diameter that changes over the length of the portion of the fluid pathway, the geometric factor is then:

$${G}_{f} = {\int }_{0}^{L} \frac{dl}{{D ( l )}^{4}}$$

In some embodiments, the portion of the fluid pathway may have a cross section that is not circular or may have a complicated inner diameter profile. The geometric factor can then be determined by measuring the flow rate (Q) at given pressure (ΔP) with known viscosity (μ) fluid:

$${G}_{f} = \frac{π Δ P}{128 μ Q}$$

The G_{f} value of the portion of the fluid pathway may be selected to reduce the maximum shear stress for each catheter gauge to be the same or less than the max shear stress of a BD 21G VACUTAINER® UltraTouch™ push button blood collection set (available from Becton, Dickinson & Company of Franklin Lakes, N.J.), which was previously considered the gold standard for blood draws. In some embodiments, G_{f} value of the portion of the fluid pathway may be selected to reduce the maximum shear stress for each catheter gauge to be the same or less than the max shear stress of a BD 25G VACUTAINER® UltraTouch™ push button blood collection set (available from Becton, Dickinson & Company of Franklin Lakes, N.J.).

In some embodiments, the fluid pathway of a catheter system, such as, for example, that of FIGS. 2-3, which include the portion of the fluid pathway, may include an entirety of a blood collection pathway through which blood flows after leaving the vasculature and into or through the blood collection device 52 during blood collection. The system geometric factor G_{fs} for the fluid pathway of the catheter system can be determined in similar fashion as the G_{f} value of the portion of the fluid pathway described earlier. In some embodiments, the system geometric factor G_{fs} with the instrument 28 at the advanced position may be equal to or more than 7.34E+06 (1/in³). In some embodiments, G_{f}s may include another value. In some embodiments, the system geometric factor G_{fs} with the instrument 28 at the advanced position may be 7.34E+06 (1/in³) plus or minus 10 percent, plus or minus 25 percent, plus or minus 50 percent, or plus or minus 75 percent. In some embodiments, G_{fs} may include another value, which may be selected based on a gauge and/or length of the catheter.

In some embodiments, the bag 22 may be constructed of a flexible and compressible material. In some embodiments, the bag 22 may include polyethylene or another suitable material. As illustrated in FIG. 3, the bag 22 may bunch or collapse in response to advancement of the instrument 28 by pinching the instrument 28 with the pincher 36 through the bag 22. In some embodiments, only the portion of the bag 22 may bunch up, such that the bag 22 does not interfere with advancement of the instrument 28.

In some embodiments, when the instrument 28 is in the retracted position, the distal end 34 of the instrument 28 may be disposed within the housing 12 to prevent contact of the instrument 28 with an external environment outside of the vascular access device 10. In some embodiments, when the instrument 28 is in the retracted position, the instrument 28 may extend through the septum 32, which may provide stability and/or guidance of the instrument 28. In some embodiments, when the instrument 28 is in the advanced position, as illustrated, for example, in FIG. 3, the distal end 34 of the instrument 28 may be disposed within the catheter assembly 66 or distal to the catheter 76, which may facilitate thrombus removal.

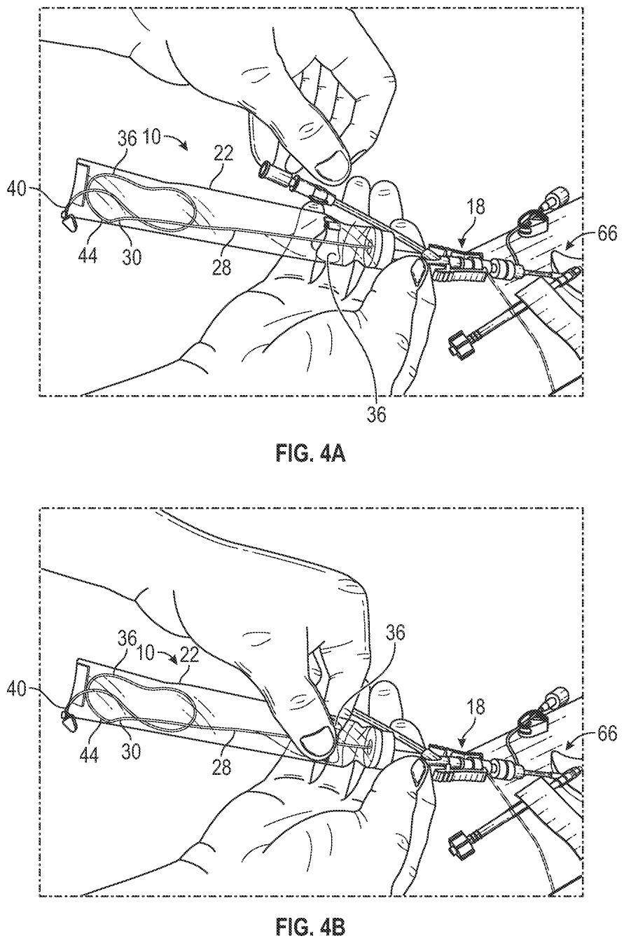

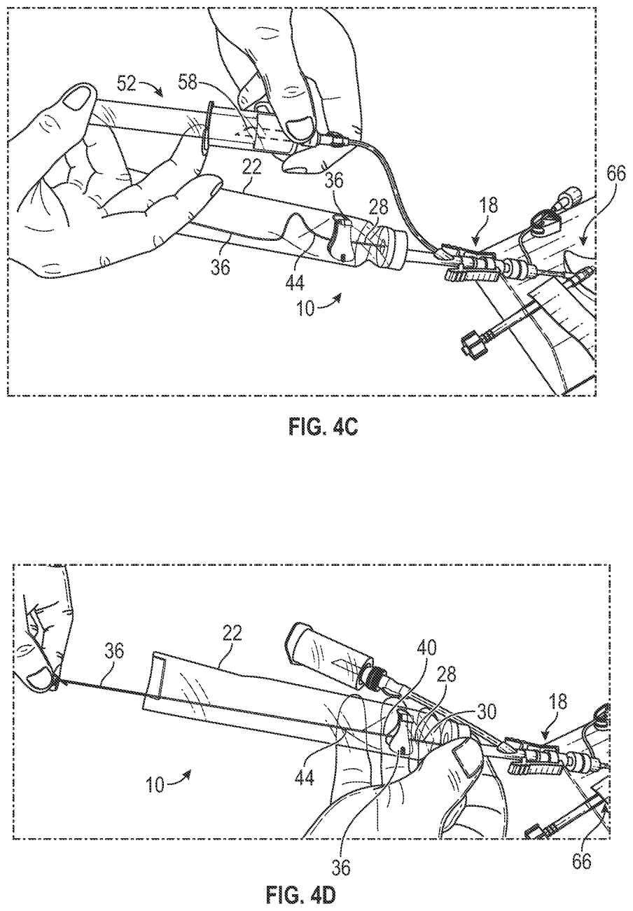

Referring now to FIGS. 4A-4D, in some embodiments, a method may include coupling the vascular access device 10 to the catheter assembly 66, as illustrated, for example, in FIG. 4A. In some embodiments, the vascular access device 10 may be coupled to the catheter assembly 66 after the catheter assembly 66 is inserted into the vasculature of the patient.

In some embodiments, the method may include pinching the instrument 28 with the pincher 36 after coupling the vascular access device 10 to the catheter assembly 66, as illustrated, for example, in FIG. 4B. In some embodiments, after pinching the instrument 28 with the pincher 36, the method may include moving the instrument 28 distally from the retracted position to the advanced position. In some embodiments, the vascular access device 10 and movement of the instrument 28 to the advanced position may facilitate patency, fluid infusion, and blood draw by preventing or delaying thrombus development and attachment to the catheter assembly 66.

In some embodiments, the method may include collecting blood through the catheter assembly 66 and the vascular access device 10 after moving the instrument 28 from the retracted position from the advanced position, as illustrated, for example, in FIG. 4C. In some embodiments, collecting blood may include inserting the BD VACUTAINER® Blood Collection Tube (available from Becton Dickinson & Company of Franklin Lakes, N.J.) into the blood collection device such that the sharp cannula 58 pierces through a stopper of the BD VACUTAINER® Blood Collection Tube. In some embodiments, blood collection may be accomplished with a syringe or another suitable blood collection device, which may be coupled to the extension tube 50 and/or the port 48.

In some embodiments, the method may include moving the instrument 28 from the advanced position to the retracted position after collecting the blood through the catheter assembly 66 and the vascular access device 10. In some embodiments, moving the instrument 28 from the advanced position to the retracted position may include pulling the pull cord 38 proximally, as illustrated in FIG. 4D, for example. In some embodiments, the pincher 36 may be used to move the instrument 28 from the advanced position to the retracted position by pinching the instrument 28.

In some embodiments, moving the instrument from the advanced position to the retracted position may include moving the distal end 34 of the instrument 28 from a position distal to the bag 22 into the bag 22. In some embodiments, the instrument 28 may not be connected to the bag 22 and may be movable with respect to the bag 22. In some embodiments, in response to the instrument 28 being in the retracted position, the distal end 34 of the instrument 28 may be disposed within the housing 12, which may prevent the instrument 28 from poking a hole in the bag 22, and/or the position indicator 60 may be disposed within the bag 22 such that the position indicator 60 is visible to the user. In some embodiments, the instrument 28 may be fully retracted when the instrument 28 is in the retracted position.

In some embodiments, after the instrument 28 is moved to the retracted position, the vascular access device 10 may be uncoupled from the catheter assembly 66. In some embodiments, the vascular access device 10 may then be disposed of, and the bag 22 may reduce an amount of disposal material significantly. In some embodiments, the vascular access device 10 may include only two molded components, the pincher 36 and the housing 12, which may reduce a cost of manufacture of the vascular access device 10.

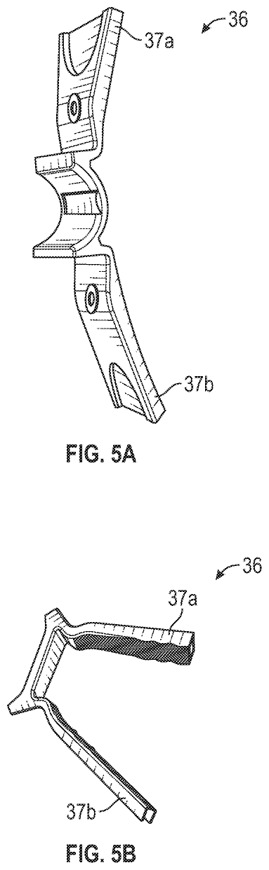

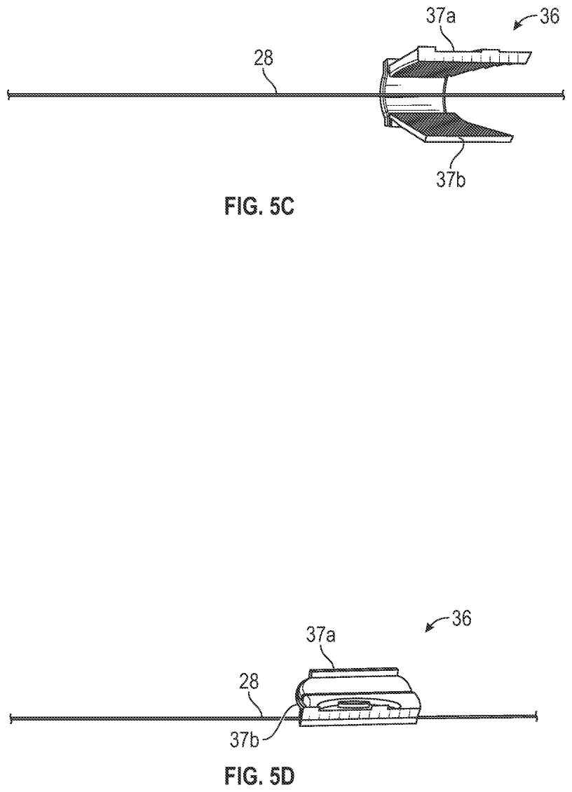

Referring now to FIGS. 5A-5D, in some embodiments, the pincher 36 may include the U-shaped body having the two arms 37a,37b configured to move towards each other to pinch the instrument 28. In some embodiments, the two arms 37a,37b may be biased away from each other and brought into contact in response to pinching of the two arms 37a,37b by the user. In some embodiments, the two arms 37a,37b may move apart from each other when the user reduces or ceases pinching, thus allowing the user to move the pincher 36 to a more proximal portion of the instrument 28 for further pinching and distal advancement of the instrument 28.

In some embodiments, the two arms 37a,37b may move apart from each other when the user reduces or ceases pinching due to the pincher 36 being constructed of a resilient material. In some embodiments, the pincher 36 may include a spring or other biasing mechanism, which may bias the two arms 37a,37b away from each other. In some embodiments, the pincher 36 may include an alligator clip or another suitable pinching device.

In some embodiments, to advance the instrument 28 distally, the user may grip the instrument 28 using the pincher 36. In some embodiments, while bunching up a portion of the bag 22, the user may then advance the instrument 28 distally through the housing 12 and/or into and/or through the catheter. In some embodiments, the user may then release the pincher 36, which allows the pincher 36 to open, and the bag 22 may be retracted proximally along a shaft of the instrument 28. In some embodiments, the user may then grip the instrument 28 again using the pincher 36 and advance the instrument 28 further in the distal direction, such as, for example, into and/or through the catheter.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A vascular access device, comprising:
a housing, comprising a distal end, a proximal end, and a lumen extending through the distal end of the housing and the proximal end of the housing, wherein the distal end of the housing comprises a connector configured to couple to a catheter assembly;
a bag coupled to the proximal end of the housing;
an instrument disposed within the bag, comprising a distal end and a proximal end; and
a pincher disposed within the bag and configured to pinch the instrument to move the instrument distally through the lumen of the housing.

2. The vascular access device of claim 1, further comprising a septum disposed within the housing or a distal end of the bag.

3. The vascular access device of claim 1, further comprising a pull cord coupled to the proximal end of the instrument and extending through an opening in the bag.

4. The vascular access device of claim 3, wherein a proximal end of the pull cord is coupled to a grip element.

5. The vascular access device of claim 1, wherein the housing comprises a port between the distal end of the housing and the proximal end of the housing, further comprising an extension tube and a blood collection device, wherein the extension tube comprises a distal end integrated with the port and a proximal end coupled to the blood collection device.

6. The vascular access device of claim 5, wherein the blood collection device comprises a sharp cannula.

7. The vascular access device of claim 1, wherein the instrument comprises a guidewire.

8. The vascular access device of claim 1, wherein the pincher comprises a U-shaped body having two arms configured to move towards each other to pinch the instrument.

9. The vascular access device of claim 8, wherein an inner surface of the two arms comprises a textured surface.

10. The vascular access device of claim 1, further comprising a position indicator disposed on the instrument, wherein the instrument is in a retracted position, wherein in response to the instrument being in the retracted position the distal end of the instrument is disposed within the housing and the position indicator is disposed within the bag.

11. The vascular access device of claim 10, further comprising a pull cord coupled to the proximal end of the instrument and extending through an opening in the bag, wherein the pull cord is compacted in response to the instrument being in the retracted position.

12. The vascular access device of claim 1, further comprising a stop disposed on the instrument with an enlarged outer diameter, wherein in response to the instrument moving distally from the retracted position to an advanced position distal to the housing, the stop contacts an inner surface of the housing.

13. A method, comprising:
coupling a vascular access device to a catheter assembly, wherein the vascular access device comprises:
a housing, comprising a distal end, a proximal end, and a lumen extending through the distal end of the housing and the proximal end of the housing, wherein the distal end of the housing comprises a connector configured to couple to a catheter assembly;
a bag coupled to the proximal end of the housing;
an instrument disposed within the bag, comprising a distal end and a proximal end; and
a pincher disposed within the bag and configured to pinch the instrument to move the instrument distally through the lumen of the housing;
pinching the instrument with the pincher after coupling the vascular access device to the catheter assembly;
after pinching the instrument with the pincher, moving the instrument from a retracted position to an advanced position, wherein in response to the instrument being in the retracted position, the distal end of the instrument is within the housing, wherein in response to the instrument being in the advanced position, the distal end of the instrument is disposed within the catheter assembly or distal to the catheter assembly.

14. The method of claim 13, wherein the housing comprises a port between the distal end of the housing and the proximal end of the housing, further comprising an extension tube and a blood collection device, wherein the extension tube comprises a distal end integrated with the port and a proximal end coupled to the blood collection device, further comprising collecting blood through the catheter assembly and the vascular access device after moving the instrument from the retracted position from the advanced position.

15. The method of claim 14, wherein the vascular access device further comprises a pull cord coupled to the proximal end of the instrument and extending through an opening in the bag.

16. The method of claim 15, wherein a proximal end of the pull cord is coupled to a grip element.

17. The method of claim 14, further comprising moving the instrument from the advanced position to the retracted position after collecting the blood through the catheter assembly and the vascular access device, wherein moving the instrument from the advanced position to the retracted position comprises pulling the pull cord proximally.

18. The method of claim 17, further comprising a position indicator disposed on the instrument, wherein moving the instrument from the advanced position to the retracted position comprises moving the distal end of the instrument from a position distal to the bag into the bag wherein the instrument is in a retracted position, wherein in response to the instrument being in the retracted position, the distal end of the instrument is disposed within the housing and the position indicator is disposed within the bag.

19. The method of claim 13, further comprising a stop disposed on the instrument with an enlarged outer diameter, wherein in response to the instrument moving distally from the retracted position to the advanced position distal to the housing, the stop contacts an inner surface of the housing.

20. The method of claim 17, wherein the instrument comprises a guidewire, wherein the housing or the bag is transparent, further comprising a position indicator disposed on the guidewire and another position indicator on the housing or the bag, wherein moving the instrument from the advanced position to the retracted position comprises moving the distal end of the guidewire from a position distal to the bag into the bag, wherein in response to the instrument being in the retracted position, the position indicator is aligned with the other position indicator.
